(19) United States
(12) Patent Application Publication
(10) Pub. No.: US 2007/0149440 A1
(43) Pub. Date: Jun. 28, 2007

(54) TRANSFERRIN AND TRANSFERRIN-BASED COMPOSITIONS FOR DIABETES TREATMENT

(75) Inventor: Zoltan Kiss, Austin, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: ESSENTIAL SKINCARE, LLC, Austin, MN (US)

(21) Appl. No.: 11/616,378

(22) Filed: Dec. 27, 2006

Related U.S. Application Data
(60) Provisional application No. 60/754,446, filed on Dec. 28, 2005.

(51) Int. Cl. A61K 38/40; A61K 31/70; A61K 31/426; A61K 31/17; A61K 31/155

(52) U.S. Cl. 514/6; 514/25; 514/594; 514/369; 514/635

(57) ABSTRACT

Embodiments of the present invention use transferrin or active derivatives to control or stabilize abnormally elevated levels of blood glucose in mammals, particularly humans. Embodiments of the invention also provide methods for enhancing survival of islet β-cells in vivo and in vitro. In other embodiments, transferrin or active derivatives are used in combination with anti-diabetic medicaments or performing an insulin elevating procedure. In yet another embodiment the invention includes administering transferrin or an active derivative to reduce weight loss induced by type 1 diabetic condition.

### CLAIM OF PRIORITY

This application claims the benefit of provisional application Ser. No. 60/754,446 filed Dec. 28, 2005, incorporated by reference herein.

### FIELD OF INVENTION

In embodiments of the invention, human transferrin is used alone or in combination with other agents to reduce blood glucose levels in type 1 and type 2 diabetic subjects as well as enhance islet survival and prevent body weight loss induced by the type 1 diabetic condition.

### BACKGROUND

Diabetes mellitus is a heterogeneous group of disorders characterized by high blood glucose levels. Type 1 or insulin-independent diabetes results from an absolute deficiency of insulin due to autoimmune destruction of the insulin-producing pancreatic P-cell islets [Atkinson, M. A. (2005), “Thirty years of investigating the autoimmune basis for type 1 diabetes,” Diabetes, 54, 1253-1263]. People who suffer from type 1 diabetes must take exogenous insulin to prevent the development of ketoacidosis. In type 2 or non-insulin-dependent diabetes mellitus, muscle, fat, and liver cells are resistant to the actions of insulin. In addition, the mechanisms that are activated in β-cells to secrete insulin to maintain blood glucose levels within a normal physiological range fail to function properly. Type 2 diabetes accounts for about 90% of all diabetes cases.

Diabetes is a potentially dangerous disease because it is associated with markedly increased incidence of coronary, cerebral, and peripheral artery disease. As a result, patients with diabetes have a much higher risk of developing other disorders such as myocardial infarction, stroke, limb amputation, renal failure, or blindness. Atherosclerotic cardiovascular disease is responsible for 80% of diabetic mortality and more than 75% of all hospitalizations for diabetic complications [Brownlee, M. (2001), “Biochemistry and molecular cell biology of diabetic complications,” Nature, 414, 813-820].

Despite large variations in carbohydrate intake with various meals, blood glucose levels remain in a narrow range between 4 and 6 mM in non-diabetic individuals. This tight control is regulated by the balanced operation of three major mechanisms. These mechanisms are: (i) glucose absorption by the intestine, (ii) glucose production by the liver, and (iii) uptake and metabolism of glucose by the peripheral tissues, mainly the skeletal muscle and fat tissue. In the skeletal muscle and fat tissue, insulin increases uptake of glucose as well as conversion of glucose to other metabolites such as glycogen and fat (mainly triglycerides). In the liver, insulin inhibits the release of glucose from glycogen and the synthesis of new glucose. Insulin is the only known hormone that can regulate all of these mechanisms needed to maintain the blood glucose level in the normal range.

Presently, the only available treatment for type 1 diabetic subjects is administration of insulin either via injection or orally. In either case, insulin must be administered daily and most often multiple times a day. Unfortunately, in some cases severe hypoglycemia may result from insulin treatment. Thus, type 1 diabetic patients would greatly benefit from the use of an anti-diabetic agent with a longer lasting effect and a better safety profile than insulin which would allow the use of insulin less frequently and at smaller doses.

In type 2 diabetic patients, insufficient control of hyperglycemia (elevated blood glucose level) gradually leads to the progression into type 1 diabetes. Several drugs in five major categories, each acting by a different mechanism, are available to control hyperglycemia. Sulfonylureas, such as, for example, glibenclamide plus nateglidine, act on the pancreatic β-cells to enhance secretion of insulin. While this therapy can decrease blood glucose levels, it has limited efficacy and tolerability. In addition, it may cause weight gain and often induces hypoglycemia. In some cases, patients become resistant to this treatment. Biguanides, such as Metformin, is thought to primarily act in the liver by decreasing glucose production. However, this agent often causes gastrointestinal disturbances and lactic acidosis that limits its use. Acarbose, an inhibitor of α-glucosidase, decreases absorption of glucose from the intestine. However, like biguanides, this agent also often results in gastrointestinal disturbances. Pioglitazone and rosiglitazone, members of the thiazolidinedione family of molecules, each regulate lipid metabolism and thus enhance the response of liver, muscle and fat tissues to the actions of insulin. However, frequent use of these drugs may lead to weight gain and may induce edema and anemia. The fifth category represents numerous insulin products with shorter and longer duration of action.

In severe cases of type 2 diabetes, insulin is used alone or in combination with the above agents. However, because each agent has either significant side effects or causes weight gain, newer approaches to control type 2 diabetes are needed. A suitable agent for treating type 2 diabetes would not have significant side effects such as induction of hypoglycemia or weight gain. It may be metabolically stable enough to allow its less frequent administration. Finally, it may be used in combination with tolerable amounts of any of the other drugs listed above to control blood glucose level in type 2 diabetic subjects.

### SUMMARY OF THE INVENTION

In an embodiment of the invention transferrin or active derivatives are used to reduce blood glucose levels in a mammal with type 1 or type 2 diabetes. In other embodiments, chromium is further included to reduce blood glucose levels in a mammal with type 1 or type 2 diabetes. In yet other embodiments of the invention, transferrin is used to enhance the survival of islet β-cells. In still other embodiments, transferrin is used to reduce body weight loss induced by type 1 diabetes.

### DETAILED DESCRIPTION OF THE INVENTION

Embodiments of the invention provide a method of controlling or stabilizing abnormally elevated levels of blood glucose in mammals, particularly humans, through administration of transferrin (TF) or an active derivative of TF. The term “abnormally elevated” refers to a mammal's blood glucose level that is greater than the normal range for that mammal. A normal blood glucose level in a human is in a range of 4-6 mM.

In one embodiment, the invention provides a method of reducing the blood glucose level in a human with type 1 or type 2 diabetes by administering a therapeutically effective amount of TF, or an active derivative. The terms, “transferrin”, “TF”, and “human TF” refer to the related group of glycosylated or non-glycosylated transferrin core proteins or fragments that are capable of lowering blood glucose level. The phrase “active derivative” refers to any of the glycosylated or non-glycosylated TF-like core proteins or fragments that are capable of lowering blood glucose level. The phrase “therapeutically effective amount” is used throughout this application to indicate a dosage that is effective in, or is targeted at, attaining or maintaining a level of glucose in a mammal's blood that is within the normal range for that mammal. The normal blood glucose level in a human is in a range of 4-6 mM.

In another embodiment, the method may include the step of administering an anti-diabetic medicament or employing an insulin elevating procedure in combination with the TF or active derivative. The phrase “in combination” refers to the use of an anti-diabetic medicament that may be administered simultaneously or separately from administration of the TF or active derivative. Examples of suitable anti-diabetic medicaments, either already in the clinical practice or in the pipeline of development, include insulin, sulfonylureas, non-sulfonylurea insulin secretogogues, biguanide, inhibitors of α-glucosidase, thiazolidinediones, α₁-acid glycoprotein (AGP), placental alkaline phosphatase, α₁-antitrypsin, and NN2211 or similar glucagon-like peptide-derived peptides or chromium.

In still another embodiment, the invention provides a treatment regimen for treating diabetes by periodically administering to a human a therapeutically effective amount of TF, or an active derivative. The term “periodically” refers to repeated administration of TF aimed at restoring or maintaining a normal level of glucose in the human's blood. The periods do not have to be uniform. The therapeutically effective amount of TF may be different at each administration depending upon the concentration of blood glucose in the human. This treatment regiment may be used to treat both type 1 and type 2 diabetic mammals, including humans. The treatment regimen may also be used in combination with administration of an anti-diabetic medicament. The treatment regimen may be effective to maintain the human's blood glucose level below about 10 mM, or between 4 mM-6 mM.

In another embodiment, the invention provides a method of enhancing the survival of islet cells that includes administering a therapeutically effective amount of TF. This method may be used particularly in type 1 diabetic patients. The islet cells may be native to the patient, or may be transplanted islet cells. TF may be used together with a growth factor, NN2211 or another glucagon-like peptide-derived peptide, betacellulin, a thiazolidinedione, or α₁-antitrypsin to promote islet cell survival.

In yet another embodiment, the invention provides a method of reducing the weight loss induced by the type 1 diabetic condition by administering a therapeutically effective amount of TF alone or together with placental alkaline phosphatase.

In another embodiment, the invention provides for the use of TF, or an active derivative, for the manufacture of a medicament for the treatment of diabetes. The medicament may include TF or an active derivative dissolved or dispersed in a suitable carrier. The medicament may also be suitable for co-administration with another anti-diabetic medicament, such as those described above.

In still another embodiment, the invention provides a method of enhancing the glucose-lowering effect of insulin by administering a therapeutically effective amount of TF.

Description of Transferrin (TF): The Active Component

TF is a glycoprotein with an approximate molecular weight of 80 kDa. One of its functions is to carry iron from the sites of intake into the systemic circulation to the cells and tissues. The properties of TF and the receptor through which TF enters the cells have been studied [Qian, Z. M., Li, H., Sun, H. and Ho, K. (2002), “Targeted drug delivery via the transferrin receptor-mediated endocytosis pathway,” Pharmacol., Rev. 54, 561-587].

TF is available in both iron-free and iron-containing forms. However, since iron-free and iron-containing TF are equally effective in reducing blood glucose level, it may be suitable to use an iron-free preparation in the embodiments of the present invention. Administration of iron-free TF carries a lower risk of causing iron overload in the patient than administration of iron-containing TF.

Preparations of TF for use in embodiments of the invention may be obtained through a variety of methods. In one embodiment, commercial TF may be used. For example, Sigma-Aldrich produces three suitable TF preparations each prepared from human blood. The first preparation is essentially iron-free Apo-TF (aTF; catalog number T 1147 according to the 2004/2005 Sigma Catalog). The second preparation is iron-containing holo-TF (hTF; catalog number T 4132; iron content: 1100-1600 μg per 1-g protein). The third preparation is practically endotoxin-free and contains relatively low amount of iron (cfTF; catalogue number, T 3309; iron content: 300-600 μg per 1-g protein). The TF protein content in these commercially available preparations is greater than 97% in the aTF and hTF preparations and greater than 98% in effF. All these preparation are considered to be highly purified. In the ef]FF protein preparation, no contaminating proteins could be detected by employing one-dimensional gel electrophoresis for protein separation and coomassie blue for protein staining.

Less pure commercial TF preparations may be further purified using one or more chromatographic steps to obtain a homogeneous TF preparations. However, TF preparations with some impurities may also be used in embodiments of the invention, so long as the given composition includes a therapeutically effective amount of TF, the impurities do not cause any significant side effect, and the impurities do not interfere with the beneficial effects of TF.

In another embodiment, TF is obtained from a raw extract of placental tissue. Since TF is a major blood protein, and placental tissue contains a significant volume of blood, human TF may be obtained by extraction from human placental tissue. One example of a suitable extraction method is a butanol extraction. Other methods of extraction from placental tissue are also suitable.

Raw extracts of blood or placenta may also be enriched using physical concentration methods in order to create a suitable preparation of TF. The concentration of TF in some raw extracts may be too low to have a blood glucose level-lowering effect when administered to a subject. Therefore, raw blood or placenta-derived TF extractions may be treated with one or more purification steps, such as solvent extraction, column chromatography separation, or other separation methods to increase the concentration of TF as compared to the concentration of TF in the raw extract. An advantage of using a purified or homogenous preparation of TF in the embodiments of the present invention is that possible side effects caused by contaminating proteins may be avoided. However, as mentioned above, impure TF may also be used in embodiments of the present invention, so long as the given composition includes a therapeutically effective amount of TF, the impurities do not cause any significant side effect, and the impurities do not interfere with the beneficial effects of TF. Since every consecutive purification step results in some loss of the protein, using a TF preparation that is less than homogeneous in some embodiments of the present invention may be more cost-effective.

In another embodiment, recombinant TF may be produced. In such an embodiment, using the corresponding cDNA sequence, the original TF or a point or deletion mutant is expressed in any suitable cell line, for example in insect cells [Tomiya, N., Howe, D., Aumiller, J. J., Pathak, M., Park, J., Palter, K. B., Jarvis, D. L., Betenbaugh, M. J. and Lee, Y. C. (2003), “Complex-type biantennary N-glycans of recombinant human transferring from Trichoplusia in cells expressing mammalian β-1,4-galactotransferase and β-1,4-N-acetylglucosaminenyltransferase II,” Glycobiology, 13, 23-34]. These and similar techniques may be used to generate, at larger scale, various active recombinant forms of TF and its active derivatives.

Methods of Use.

The results presented in the Examples section imply that TF can act via three related mechanisms in diabetic animals, and by implication in diabetic humans as well. The first mechanism is lowering of blood glucose level in type 2 diabetic subjects by enhancing the efficacy of insulin. The second mechanism is protection of islets in type 1 diabetic subjects, which is expected to result in more insulin production and better glucose control. The third mechanism is reduction or prevention of body weight loss in type 1 diabetic subjects. For all these applications, in one method TF may be injected into the patient. Any suitable injection method, such as intravenous, intraarterial, intraperitoneal, subcutaneous, intradermal, and intramuscular may be used. In another embodiment, osmotic minipumps or any other types of pumps that can be inserted under the skin and provide for controlled protein release are also suitable. In yet another embodiment, TF may be prepared as a dry powder and administered, similar to certain solid insulin products such as Exubera [White, S., Bennett, D. B., Cheu, S., Conley, P. W., Guzek, D. B., Gray, S., Howard, J., Malcolmson, R., Parker, J. M., Roberts, P., Sadrzadeh, N., Schumacher, J. D., Seshadri, S., Sluggett, G. W., Stevenson, C. L. and Harper, N. J. (2005), “EXUBERA: Pharmaceutical development of a novel product for pulmonary delivery of insulin,” Diabetes Technol. Ther., 7, 896-906], via inhalation using a suitable inhalation device. The technology required for the production of an inhalation TF preparation, including steps such as chemical stabilization of the protein, dry powder formulation, powder filling and packaging, and a mechanical device for powder dispersal and reliable dosing, is available. However, considering that the half-life time of TF in the circulation is relatively long (˜7 days) which allows once or twice a weekly application, the most common delivery method in some embodiments of the invention is likely to be by injection.

The maximum protein content in the blood depends on the size of the protein and the type of injection used. In case of a relatively smaller peptide (less than 5 kDa), intravenous injection into mice resulted in about 40-times higher protein concentration in the blood compared to subcutaneous injection [Szepeshazi, K., Schally, A. V., Halmos, G., Lamharzi, N., Groot, K. and Horvath, J. E. (1997), “A single in vivo administration of bombesin antagonist RC-3095 reduces the levels and mRNA expression of epidermal growth factor receptors in MXT mouse mammary cancers,” Proc. Natl. Acad. Sci. U.S.A., 94, 10913-10918]. In case of Tf, an 80 kDa protein, the initial difference between the two application methods maybe even greater, because uptake of a larger protein through the endothelium is always a less efficient process than uptake of a smaller protein. However, since the half-life of TF in the human blood is about 7 days, one can expect that after several hours the blood level of TF will achieve an equilibrium value that is similar in case of both intravenous or subcutaneous. Accordingly, the specific method of injection may be decided based on how fast the action needs to be. For example, if there is a need for a very rapid decrease in the blood glucose level, then intravenous application may be the most preferred method. However, if longer-term stabilization of blood glucose or less use of insulin is the goal, then subcutaneous application may be appropriate. Subcutaneous or even intradermal application may also be a preferred application for preventing body weight loss in type 1 diabetic subjects.

In one embodiment of the invention, TF is prepared for injection by adding TF to physiological saline or dissolving TF in any other biologically compatible solution, or enclosed in immunoliposomes or other delivery systems. Systemic treatment may reduce a patient's normal range of blood glucose level to less than about 10 mM, or less than about 8 mM, and possibly within the normal range of about 4-6 mM. The dosage and the number of treatments needed to achieve the blood glucose-lowering effect may be dependent on a number of factors such as the severity of diabetes, the tolerance of the individual patient, and the chosen injection method. For example, in one embodiment, the patient may receive a dose of between about 0.01 to 5-g/m² once daily via an injection route. In another embodiment, the patient may receive a dose of between about 0.025 to 2.0-g/m² once daily. In still another embodiment, the patient may receive a dose of between about 0.05 to 1.0-g/m² once or twice weekly. In another embodiment, a patient may receive a dose of between 50 to 400-mg TF per 100-kg human. In still another embodiment, a patient may receive a dose of about 200-mg TF per 100-kg human.

Since entry of TF into the blood supply and then into the target tissues requires some time, for rapid action it may be suitable to administer TF intravenously to the patient just prior to an expected glucose load. In one embodiment, TF may be administered intravenously to a patient 5-10 minutes prior to an expected glucose load. In another embodiment, TF may be administered subcutaneously or intravenously to a patient about 0.5-2 hours prior to an expected glucose load. In yet another embodiment, TF may be administered subcutaneously or intravenously about 24 hours prior to an expected glucose load.

If TF is co-administered with other anti-diabetic medicaments or with an insulin elevating procedure such as islet transplantation, then TF may be administered before, after, or with the other medicaments or procedures. For example, if glycemic control is required rapidly, insulin may be administered before administration of TF or an active derivative. In this instance, TF may provide a long-lasting blood glucose level-lowering effect and prevent the need for administering additional insulin. However, if the blood glucose level in a patient is less severe, for example if it is below 10 mM, administration of TF alone or TF with other anti-diabetic agents or insulin elevating procedures may provide appropriate glycemic control. If TF is used continuously for treatment, other anti-diabetic medicaments (as listed later) may be administered in addition to TF to provide finely controlled glycemic levels in the blood.

The duration of treatment with TF may be varied according to the needs of the patient. In one embodiment, TF is used to provide short-term glycemic control and the duration of the treatment lasts for about one month. Since a sustained increase in the blood concentration of TF may offset the required balance of iron metabolism, prolonged durations of treatment with TF may result in negative physiological consequences for the patient. However, significant increases in the amount of TF in the blood for less than one month are not expected to cause side effects. Also, there are methods available to compensate for the potential imbalance in iron metabolism caused by longer-term TF administration. Eventually a physician will determine the duration of treatment with TF on an individual basis based on other circumstances. These circumstances may include, for example, the patient's tolerance for TF, the patient's history of hypoglycemia when treated with insulin, the patient's resistance to other anti-diabetic medicaments, and the patient's iron balance. The doctor may also decide to remove the patient from treatment with TF for a period of time to reduce the likelihood that the patient will develop resistance to TF. Furthermore, a doctor may decide to reduce the likelihood of resistance to other anti-diabetic medicaments by periodically treating a patient with TF, and then resuming treatment with other anti-diabetic medicaments such as insulin.

Co-Administration of TF with Anti-Diabetic Medicaments to Decrease Blood Glucose Level in Type 2 Diabetic Subjects.

TF may be co-administered with other anti-diabetic medicaments, already in the clinical practice or in the pipeline of development, to decrease blood glucose level in type 2 diabetic subjects. Suitable clinical anti-diabetic medicaments for co-administration with TF include insulin, sulfonylureas such as, for example, Glyburide, Glipizide, or Glimepride, metiglinides (non-sulfonylurea secretagogues) such as nateglidine and repaglidine, biguanides such as metformin, inhibitors of α-glucosidase such as acarbose and miglitol, and thiazolidinediones such as pioglitazone and rosiglitazone. Sulfonylureas and metiglinides act by stimulating insulin secretion, while the others act by different mechanisms including enhancement of insulin effects. Since TF appears to act by enhancing the effects of insulin, it is expected that it will work the best together with sulfonylureas and metiglinides that increases insulin level in the circulation. TF is not expected to work well with other agents that also enhance the actions of insulin like thiazolidinediones do.

Incretins, such as glucagon-like peptide (GLP)-1 and glucose-dependent insulinotropic polypeptide (GIP) and particularly the long-acting GLP-1 derivative NN2211 may be used in some embodiments of the invention. Incretins are generally considered to regulate blood glucose levels mainly via stimulation of insulin release from the islets [Holst, J. J. and Gromada, J. (2003) Role of incretin hormones in the regulation of insulin secretion in diabetic and nondiabetic humans. Am. J. Physiol. Endocrinol. Metab. 287, E199-E206], although they also exert other important effects including suppression of glucose production in the liver [Prigeon, R. L., Quddusi, S., Paty, B. and D'Alessio, D. A. (2003), “Suppression of glucose production by GLP-1 independent of islet hormones: a novel extrapancreatic effect,” Am. J. Physiol. Endocrinol. Metab., 285, E701-E707], induction of weight loss [Larsen, P. J., Fledelius, C., Knudsen, L. B. and Tang-Christensen, M. (2001), “Systemic administration of the long-acting GLP-1 derivative NN2211 induces lasting and reversible weight loss in both normal and obese rats,” Diabetes, 50, 2530-2539], and stimulation of pancreatic β-cell proliferation [Buteau, J., Foisy, S., Rhodes, C. J., Carpenter, L., Biden, T. J. and Prentki, M. (2001), “Protein kinase Cζ activation mediates glucagon-like peptide-1-induced pancreatic β-cell proliferation,” Diabetes, 50, 2237-2243]. Incretins represent another group of anti-diabetic drugs that are likely to enhance the effects of TF on blood glucose levels.

In some embodiments, any anti-diabetic agent, approved or in the experimental phase, together with TF is used to control blood glucose level in type 2 diabetic subjects. Such agents are α₁-acid glycoprotein [PCT Publication No. WO 2005/117937, published Dec. 15, 2005 and U.S. patent application Ser. No. 11/241,068, filed May 27, 2005, entitled “Alpha-1-acid glycoprotein for the treatment of diabetes”; inventor, Zoltan Kiss] and placental alkaline phosphatase [PCT Publication No. WO 2004/054609, published Jul. 1, 2004, titled “Placental alkaline phosphatase to control diabetes”; inventor, Zoltan Kiss], each incorporated by reference herein. In pre-clinical glucose tolerance tests both proteins effectively decreased blood glucose levels.

Transferrin (TF) is able to bind not only iron but also chromium. It is postulated that TF-bound chromium is carried inside the cells by TF receptor-mediated endocytosis. Once inside, chromium can enhance the effect of insulin on glucose transport in insulin-sensitive tissues such as skeletal muscle [Cefalu, W. T. and Hu, F. B. (2004), “Role of chromium in human health and in diabetes.” Diabetes care, 27, 2741-2751]. Accordingly, one mechanism by which subcutaneously injected TF could decrease blood glucose level is via binding free chromium in the circulation and/or the interstitial space and then transporting chromium inside the insulin-sensitive tissues.

Some embodiments of the invention use TF in combination with chromium or derivatives thereof When CrCl₃ was co-injected with a very low concentration of TF that alone had only marginal effect, chromium enhanced the ability of TF to decrease blood glucose level.

TF is co-administered with CrCl₃ which is the least toxic form of chromium; it is essentially non-toxic up to a daily intake of 1,000 μg. The recommended dose of CrCl₃ when administered together (in the same solution) with TF is 50-200 μg.

In some embodiments, TF is iron-free, so one molecule of protein will bind at least one molecule of chromium.

The combination of TF and chromium may be administered once a week or twice a week or as frequently as necessary to decrease blood glucose levels.

2. Co-Administration of TF with Agents That Enhance Islet Survival.

In the Examples, treatment of mice with TF prior to the destruction of islet cell function by streptozotocin, but not after streptozotocin treatment, can partially normalize blood glucose levels. Because TF does not enhance insulin secretion, TF partially protects the islets from the destructive effects of streptozotocin. The mechanism is supported by the finding that in vitro TF promoted survival of NIT-1 mouse islet β-cells in serum-free medium. Serum-free medium and streptozotocin commonly generate oxygen free radicals that are responsible for the induction of death of islet β-cells.

Accordingly, in one embodiment of the invention, TF enhances survival of islet cells in vivo, particularly in diabetic patients, in the absence of other treatment. By enhancing the survival of islet cells, TF increases the amount of secreted insulin and reduces or prevents abnormally elevated levels of blood glucose. The islet cells may be native to the patient, or may be transplanted islet cells. In this embodiment, the administration routes, doses, and treatment schedules may be similar to those described above.

The ability of TF to promote islet β-cell survival may also be used to normalize blood glucose level in type 1 diabetic subjects by co-administering TF with other agents that, similar to TF, partially improve islet survival in vivo. The list of such islet survival-promoting agents include α₁-antitrypsin [Lewis, E. C., Shapiro, L., Bowers, O. J. and Dinarello, C. A. (2005), “α₁-antitrypsin monotherapy prolongs islet allograft survival in mice,” Proc. Natl. Acad. Sci. USA, 102, 12153-12158], the GLP-1 derivative NN2211 [Rolin, B., Larsen, M. O., Gotfredsen, C. F., Deacon, C. F., Carr, R. D., Wilken, M. and Knudsen, L. B. (2002), “The long-acting GLP-1 derivative NN2211 ameliorates glycemia and increases β-cell mass in diabetic mice,” Am. J. Physiol. Endocrinol. Met., 283, E745-E762], amino acids like leucin, growth factors such as insulin like growth factor-1, insulin like growth factor-2, insulin, growth hormone, platelet-derived growth factor, placental lactogene, gastrin, prolactin, hepatocyte growth factor, fibroblast growth factor, islet neogenesis-associated peptide, transforming growth factor-α, glucagon, nerve growth factor, and vascular endothelial growth factor [Nielsen, J. H., Galsgaard, E. D., Moldrup, A., Friedrichsen, B. N., Billestrup, N., Hansen, J. A., Lee, Y. C and Carlsson, C., (2001), “Regulation of β-cell mass by hormones and growth factors,” Diabetes, 50 (Suppl. 1): S25-S29], thiazolidinedione compounds like pioglitazone [Ishida, H., Takizawa, M., Ozawa, S., Nakamichi, Y., Yamaguchi, S., Katsuta, H., Tanaka, T., Maruyama, M., Katahira, H., Yoshimoto, K., Itagaki, E. and Nagamatsu, S. (2004), “Pioglitazone improves insulin secretory capacity and prevents the loss of β-cell mass in obese diabetic db/db mice: Possible protection of β cells from oxidative stress,” Metabolism, 4, 488-494], or betacellulin [Li, L., Seno, M., Yamada, H. and Kojima, I. (2003), “Betacellulin improves glucose metabolism by promoting conversion of intraislet precursor cells to β-cells in streptozotocin-treated mice,” Am. J. Physiol. Endocrinol. Metab., 285, E577-E583].

TF in the concentration range of about 0.1-10 μg per ml promotes cell migration [Carlevaro, M. F., Albini, A., Ribatty, D., Gentili, C., Benelli, R., Cermelli, S., Cancedda, R. and Cancedda, D. (1997), “Transferrin promotes endothelial cell migration and invasion: Implication in cartilage neovascularization,” J. Cell Biol., 136, 1375-1384]. TF at 50 μg per ml concentration also maximally stimulates cell proliferation [Ekblom, P., Thesleff, I., Saxen, L., and Timpl, R. (1983), “Transferrin as a fetal growth factor: Acquisition of responsiveness related to embryonic induction,” Proc. Natl. Acad. Sci. USA, 80, 2651-2655]. However, the effects of TF on cell survival, and in particular on islet β-cell survival, are less studied. Since cell proliferation and cell survival involve different cellular mechanisms, it cannot be assumed that an agent that stimulates cell survival will also promote cell proliferation. The finding that TF enhances islet β-cell survival allows its application in vitro to supplement cell culture media used during the isolation and storage of human islets destined for transplantation. Transplantation of islets is a recently developed experimental procedure being performed to cure type 1 diabetic patients. However, a large fraction of islets do not survive the isolation procedure. Inclusion of TF in the isolation and storage media is expected to increase the number of surviving islets and thereby the number of recipients of islet transplants. Considering its effective concentrations in various cell culture media, TF may be used in the concentration range of about 0.1-50 βg per ml to enhance the survival of human islets during the isolation procedure.

Application of TF to Reduce Weight Loss in Type 1 Diabetic Subjects.

TF may also be administered to reduce weight loss induced by type 1 diabetic condition. In the Examples, administration of TF was effective in preventing streptozotocin-induced loss of weight in type 1 diabetic animals. Since streptozotocin-induced loss of body weight is attributed to type 1 diabetic condition, the results imply that TF can also prevent or reduce body weight loss in subjects who develop type 1 diabetic states. In this embodiment, the administration routes, doses, and treatment schedules may be similar to those described above.

In other embodiments of the invention, TF may be used in combination with insulin or placental alkaline phosphatase for most effective control of body weight in type 1 diabetic subjects. [U.S. Pat. No. 7,048,914 B2, entitled “Placental alkaline phosphatase to control diabetes”; issued May 23, 2006; inventor, Zoltan Kiss].

### EXAMPLES

### Example 1

### Determination of Blood Glucose in Mice in Glucose Tolerance Tests

In each relevant example, C57/Black female mice, weighing 22-23 g and fasted for 16 hours before being administered glucose intraperitoneally (3 g/kg) were used. None of the animals received any food during the experiment in addition to glucose. The mice received intraperitoneal injections of 0.05-1.5 mg/mouse of various preparations of human TF available from Sigma-Aldrich. Blood samples were taken from the eyes (canthus), and glucose concentrations in whole blood samples were immediately measured with the fast Glucose C test available from Wako Chemicals USA Inc. (Richmond, Va.). Unless indicated otherwise, each treatment group included six animals. The data are the mean±std. dev. of 6 determinations, i.e. one determination with each of the six animals.

### Example 2

### Comparison of Effects of Human apo-TF (aTF) and holo-TF (hTF) on Blood Glucose Level

In this experiment, 0.5 mg/mouse of human aTF (catalog number T 1147 according to the 2004/2005 Sigma Catalog), or 0.5 mg/mouse hTF (catalog number T 4132 according to the 2004/2005 Sigma Catalog) was administered to the mice 24 hours prior to a glucose injection. The blood glucose levels of the mice were then measured over a period of time. The results are shown in TABLE 1. In untreated mice, injection of glucose resulted in 7- to 8-fold elevation of blood glucose level (7.9 mM) by the 30th minute that declined to the 2-3 mM range 120-180 minutes after glucose administration. Both aTF and hTF suppressed the elevation of blood glucose level as compared to the untreated animals. Furthermore, both aTF- and hTF-treated animals had blood glucose levels that remained between 2-3 mM 30 to 180 minutes after the administration of glucose. This indicates that the glucose lowering effect of TF is stable and unlikely to cause serious hypoglycemia. The results also show that aTF and hTF are similarly effective in lowering blood glucose indicating that both iron-free TF (aTF) and iron-containing TF (hTF) are suitable in embodiments of the present invention. Furthermore, the fact that TF was effective in maintaining normal blood glucose levels even though it was administered 24 hours prior to the glucose load indicates that TF has long-lasting effects on blood glucose level.

TABLE 1
Both aTF and hTF prevents rise in blood glucose levels in mice
during glucose tolerance test.

| | Blood glucose level (mM) | | | | |
|---|---|---|---|---|---|
| Addition | 0 min. | 30 min. | 60 min. | 120 min. | 180 min. |
| None | 1.1 ± 0.2 | 7.9 ± 0.7 | 6.1 ± 0.6 | 3.0 ± 0.2 | 2.3 ± 0.3 |
| aTF | 1.0 ± 0.3 | 2.9 ± 0.3 | 2.6 ± 0.3 | 2.6 ± 0.4 | 2.3 ± 0.5 |
| hTF | 1.3 ± 0.2 | 3.0 ± 0.2 | 2.7 ± 0.3 | 2.2 ± 0.2 | 2.0 ± 0.2 |

### Example 3

### Dose- and Time-Dependent Effects of Endotoxin-Free Homogeneous TF (efTF) on Blood Glucose Level

In this experiment, 0.2, 0.5, and 1.5 mg/mouse doses of endotoxin-free human TF (efTF) available from Sigma-Aldrich, Inc. (catalogue number, T 3309 according to the 2004/2005 Sigma Catalog) were administered to the mice 2 hours and 24 hours prior to the glucose load. The blood glucose levels of the mice were then measured over a period of time. The results are shown in TABLE 2. In the untreated group, the injection of glucose resulted in about a 5-fold elevation of blood glucose level (6.7 mM) by 30 minutes that then declined to the 2.5-3 mM range 120-180 minutes after the glucose injection. However, in the groups treated with efTF, the blood glucose level remained low in the 1.7-2.9 mM range during the whole observation period. Furthermore, as shown in Table 2, efTF provided similar effects both at the 0.2 mg/mouse dose and at the 1.5 mg/mouse dose indicating that overdosing of TF is unlikely to be a problem. efTF was also shown to be as effective as the aTF and hTF preparations indicating that the active component in each preparation was TF and not a contaminant. Finally, since there was no difference in the effects when TF was added 2 hours or 24 hours prior to the glucose load, these results indicate that TF acts rapidly on blood glucose level and its effects are long lasting.

TABLE 2
efTF decreases blood glucose levels in mice during glucose tolerance
test.

| | Blood glucose level (mM) | | | | |
|---|---|---|---|---|---|
| Addition | 0 min. | 30 min. | 60 min. | 120 min. | 180 min. |
| None | 1.4 ± 0.4 | 6.7 ± 0.5 | 4.9 ± 0.4 | 2.9 ± 0.2 | 2.5 ± 0.3 |
| efTF, | 1.3 ± 0.3 | 2.8 ± 0.3 | 2.5 ± 0.2 | 2.3 ± 0.4 | 2.0 ± 0.2 |
| 0.2 mg, 2 hrs | | | | | |
| efTF, | 1.4 ± 0.3 | 2.9 ± 0.2 | 2.5 ± 0.3 | 2.0 ± 0.3 | 1.8 ± 0.2 |
| 0.5 mg, 2 hrs | | | | | |
| efTF, | 1.6 ± 0.3 | 2.5 ± 0.4 | 2.2 ± 0.3 | 2.1 ± 0.2 | 1.9 ± 0.4 |
| 1.5 mg, 2 hrs | | | | | |
| efTF, | 1.4 ± 0.2 | 2.5 ± 0.3 | 2.2 ± 0.2 | 1.9 ± 0.1 | 1.7 ± 0.4 |
| 0.5 mg, | | | | | |
| 24 hrs | | | | | |

### Example 4

### Comparison of the Effects of Lower and Higher Doses of efTF on Blood Glucose Level During Glucose Tolerance Test

In this experiment, 0.05, 0.1, 0.2, and 0.5 mg/mouse doses of endotoxin-free human TF (efTF) available from Sigma-Aldrich, Inc. (catalogue number, T 3309 according to the 2004/2005 Sigma Catalog) were administered to mice 2 hours prior to the glucose load. The data, shown in TABLE 3, indicate that while efTF was effective at the 0.05 mg/mouse dose in decreasing blood glucose levels, this dose was less effective than the 0.1 mg/mouse dose and larger doses of efTF. This suggests that a dose of about 200-400 mg TF per 100 kg human may be sufficient to normalize blood glucose levels in diabetic patients. It was also observed that the lowest dose of TF maintained the blood glucose levels after 120 and 180 minute of glucose administration when there was very little change in blood glucose level in the control animals. This indicates that the lowest effective dose of TF will not cause hypoglycemia.

TABLE 3
Relatively low doses of efTF efficiently decrease blood glucose
during glucose tolerance test.

| | Blood glucose level (mM) | | | | |
|---|---|---|---|---|---|
| Addition | 0 min. | 30 min. | 60 min. | 120 min. | 180 min. |
| None | 1.5 ± 0.2 | 6.8 ± 0.5 | 4.7 ± 0.4 | 3.0 ± 0.3 | 2.5 ± 0.4 |
| efTF, | 1.6 ± 0.3 | 3.7 ± 0.4 | 3.1 ± 0.2 | 2.6 ± 0.2 | 2.0 ± 0.1 |
| 0.05 mg | | | | | |
| efTF, 0.1 mg | 1.4 ± 0.3 | 3.1 ± 0.3 | 2.6 ± 0.3 | 2.2 ± 0.4 | 1.7 ± 0.2 |
| efTF, 0.2 mg | 1.5 ± 0.4 | 2.6 ± 0.3 | 2.1 ± 0.4 | 1.8 ± 0.2 | 1.6 ± 0.1 |
| efTF, 0.5 mg | 1.3 ± 0.2 | 2.7 ± 0.2 | 2.4 ± 0.2 | 2.0 ± 0.1 | 1.7 ± 0.2 |

### Example 5

### efTF Reduces Blood Glucose Level in Streptozotocin (STZ)-Treated Mice if Administered Before STZ

In this experiment, mice were administered STZ and also treated with efTF to determine if TF could be used to prevent an increase in blood glucose levels in type 1 diabetic patients. Treatment of mice with 200-250 mg/kg STZ is known to destroy the islet cells of mice, thus reducing the amount of insulin produced by the animals. Therefore, STZ administration to mice is a widely accepted experimental model for type 1 diabetes.

On day 0, day 3, day 5, day 7 and day 9, one group of mice (efTF+STZ-treated) was treated with 1.5 mg efTF per mouse. On day 1, this group was administered 250 mg/kg of STZ, 24 hours after the day 0 administration of efTF. A second group of mice (STZ-treated) was treated with only STZ on day 1. A third group of mice (STZ-treated+efTF) was treated with STZ on day 1, followed by treatments with 1.5 mg of efTF on days 3, 5, 7, and 9. Finally, a fourth group of mice (Untreated) was not treated with either efTF or STZ. In this experiment, each treatment group included 6 animals. Accordingly, data are the mean±std. dev. of 6 determinations, i.e. one determination with each of the 6 animals. As in previous experiments, the blood glucose levels were measured at various time intervals. The data, shown in TABLE 4, indicates that in STZ-treated animals the blood glucose level increased by more than 3-fold by day 3. However, in STZ-treated animals that were treated with TF prior to an administration of STZ (efTF+STZ-treated), the blood glucose level was comparatively lower on each day when the blood glucose level was measured. As it is also shown in TABLE 4, the effect of TF remained practically unchanged between day 9 and day 11, even though the last dosage of TF was administered on day 9. In addition, although the data is not shown, pre-treatment of mice with TF for 24 hours failed to significantly change blood glucose levels indicating that TF did not influence blood glucose level in the absence of STZ.

This experiment was also used to determine whether TF acts by enhancing the survival of insulin-producing islet cells. As shown in TABLE 4, the blood glucose levels of mice treated with efTF only after STZ (STZ-treated+efTF) were similar to the blood glucose levels in the group of mice with model type 1 diabetes (STZ-treated). As stated above, the group of mice that was treated with efTF before STZ had comparatively lower blood glucose levels than the animals that were treated with efTF only after STZ. Since efTF did not effect the blood glucose levels of mice whose insulin producing islet cells were destroyed by administration of STZ, these data indicate that TF reduces blood glucose level in vivo in part by enhancing the survival of insulin-producing islet cells. This example also suggests that TF may be used in vitro to enhance the survival of isolated islet cells.

TABLE 4
efTF partially counteracts STZ-induced increase in blood glucose
level.

| | Blood glucose level (mM) | | | | |
|---|---|---|---|---|---|
| Addition | Day 1 | Day 3 | Day 5 | Day 8 | Day 11 |
| efTF + | 4.1 ± 0.3 | 9.2 ± 1.5 | 8.6 ± 1.1 | 8.2 ± 0.6 | 9.0 ± 1.7 |
| STZ-treated | | | | | |
| STZ-treated | 4.5 ± 0.4 | 15.6 ± 1.2 | 16.4 ± 1.6 | 16.7 ± 0.9 | 16.1 ± 1.3 |
| STZ- | 3.9 ± 0.7 | 14.7 ± 1.4 | 15.2 ± 0.8 | 15.1 ± 1.3 | 15.9 ± 1.1 |
| treated + | | | | | |
| efTF | | | | | |
| Untreated | 4.4 ± 0.6 | 4.8 ± 0.4 | 4.9 ± 0.7 | 4.6 ± 0.3 | 4.3 ± 0.5 |

### Example 6

### efTF Reduces STZ-Induced Body Weight Loss

In this example, the body weights of the animals from Example 4 were measured. As shown in TABLE 5, the body weight of STZ-treated animals decreased from 29.4±0.38 to 19.1±0.92 grams over the 21 day observation period, while the body weight of mice treated with STZ and efTF decreased from 28.1±0.53 to only 27.3±1.49 grams. Therefore, in this animal model of type 1 diabetes, efTF protects against weight loss over an extended period of time. This indicates that TF may be used to reduce the weight loss in type 1 diabetes patients.

TABLE 5
efTF partially counteracts STZ-induced weight loss.

| | Weight (in grams) | | | | | | |
|---|---|---|---|---|---|---|---|
| Addition | Day 0 | Day 3 | Day 5 | Day 7 | Day 9 | Day 11 | Day 21 |
| Untreated | 29.2 ± 0.61 | 28.7 ± 0.80 | 29.1 ± 0.56 | 29.3 ± 0.71 | 30.1 ± 0.62 | 30.4 ± 0.71 | 31.6 ± 1.02 |
| STZ-treated | 29.4 ± 0.38 | 25.7 ± 0.26 | 23.4 ± 0.8 | 23.1 ± 0.96 | 22.3 ± 1.05 | 21.7 ± 0.72 | 19.1 ± 0.92 |
| efTF + STZ- | 28.1 ± 0.53 | 28.0 ± 1.43 | 27.2 ± 0.85 | 27.0 ± 1.58 | 27.1 ± 1.41 | 26.9 ± 1.55 | 27.3 ± 1.49 |
| treated | | | | | | | |

### Example 7

### Prevention of Serum-Free Medium-Induced Death of NIT-1 Mouse Islet Cells TF

NIT-1 islet β-cells were obtained from American Type Culture Collection (ATCC CRL-2055). These cells were originally isolated from transgenic NOD mouse carrying SV 40 large T antigen gene on a rat insulin promoter. NIT-1 cells exhibit the ultrastructural features typical of differentiated β-cells, but upon prolonged cultivation they can spontaneously develop beta adenomas. Practically all cells in this cell population contain and secrete insulin, while at the same time they retained their ability to proliferate in the presence of an appropriate stimulus. The cells, maintained in Ham's F12K medium containing 10% heat-inactivated dialyzed fetal bovine serum, were used between passages 25-28.

Cells were seeded into 96-well plates at 8,000 cells/well in 10% serum-containing medium. After 24 hours, the medium was changed for serum-free medium, followed by no addition (None, 72 hrs) or addition (within 2-3 hours) of 20 μg/ml of commercial TF (catalog number: T 3309 according to the 2004/2005 Sigma-Aldrich Catalog), 1% fetal bovine serum (FBS), or 20 μg/ml of commercial TF+1% FBS. The TF stock solution was made up in Ham's F12K medium and added in 10-μl volume to the incubation medium (final incubation volume: 110-μl). Fetal bovine serum was from Sigma Aldrich and was also added, after dilution with the Ham's F12K medium, in 10-μl volume to the incubation medium. Treatments were performed for 72 hours, followed by the MTT assay to determine the relative number of viable cells. The MTT assay was also performed on the same day when the treatments were started (0 hour).

The MTT calorimetric assay is based on the ability of living cells, but not dead cells, to reduce 3-(4,5-dimethyl thiazol-2-yl)-2,5-diphenyltetrazolium bromide. [Carmichael, J, De Graff, W. G., Gazdar, A. F., Minna, J. D. and Mitchell, J. B. (1987), “Evaluation of tetrazolium-based semiautomated calorimetric assay: Assessment of chemosensitivity testing,” Cancer Res., 47, 936-942], which is hereby incorporated by reference. This is one of the most widely used detection techniques. In this assay, absorption is measured at wavelength=540, indicated in the TABLE 6 below as A₅₄₀. Higher A₅₄₀ values mean proportionally higher numbers of viable cells. Accordingly, by providing information about the portion of viable cells, the MTT assay is suitable to assess the proliferation and/or survival rates in the control and treated cell cultures.

The results, expressed as mean values±std. dev. of 8 determinations (in 8 separate wells) for each treatment, are shown in TABLE 6. Clearly, most islet cells died after incubating them in serum-free medium for 72 hours. Treatment with TF partially prevented the death of β-cells. At the low (1%) concentration used, fetal bovine serum had relatively small effect on cell survival. However, TF and serum together not only prevented cell death but actually slightly enhanced cell numbers. These data indicate that 1% serum does not contain enough growth factors to significantly prevent cell death in the absence of TF. However, the growth factors present in 1% serum can completely prevent cell death in the presence of TF. The synergistic effects between TF and growth factors in the serum on β-cell survival may explain why TF was capable of decreasing blood glucose level in STZ-treated mice and these effects may be used to promote β-cell survival in vitro during islet isolation.

TABLE 6
Protective effects of TF and FBS against serum-free medium-induced
death of NIT-1 cells.

| Additions | Relative cell number: A₅₄₀ |
|---|---|
| None, 0 hr | 1.214 ± 0.090 |
| None, 72 hrs | 0.297 ± 0.069 |
| TF, 20 μg/ml; 72 hrs | 0.802 ± 0.058 |
| FBS, 1%; 72 hrs | 0.458 ± 0.067 |
| TF, 20 μg/ml + FBS, 1%; 72 hrs | 1.559 ± 0.112 |

### Example 8

### Determination of Blood Insulin Levels in vivo

An insulin assay kit, including rat/mouse-specific insulin antibody and ¹²⁵I-insulin as key components, was purchased from DRG Instruments GmbH (Marburg, Germany; catalog number: EIA-2048).

The principle of the procedure: A radioimmunoassay method was used to determine the amount of insulin in the blood. In radioimmunoassay, known fixed concentrations of labeled tracer antigen (like labeled insulin) and antiserum are incubated such that the concentration of antigen bound to the antibody is limited. If unlabeled antigen is added to this system, there is a competition between labeled and unlabeled antigen for the fixed number of binding sites on the antibody. Thus, the amount of antibody-bound labeled antigen will decrease proportionally with increased concentration of unlabeled antigen. In practice, the unlabeled antigen is present in the biological sample, and the purpose of the assay is to determine its concentration. This can be done by separating antibody-bound from free tracer (labeled antigen) and determining radioactivity in either or both fractions. A calibration or standard curve is set up with increasing concentrations of standard unlabeled antigen (which is insulin in the present case), and from this curve the amount of antigen in the unknown sample can be calculated. Accordingly, the four basic components for the radioimmunoassay system are: a specific antiserum to the antigen to be determined, a radiolabeled antigen, a method to separate antibody-bound and free labeled antigen, and an instrument to perform counting of radioactivity.

Preparation of Plasma:

Blood was collected by venipuncture into tubes containing heparin as anticoagulant, and then the plasma fraction was separated. The samples were held at −20° C. but prior to the assay it was brought to room temperature and mixed by vortexing immediately before adding to the assay tubes.

The Assay Procedure:

On the first day 0.3-ml, 0.2-ml, and 0.1-ml of assay buffer is transferred to non-specific binding (NSB) tubes (3-4), reference (Bo) tubes (5-6) and to “sample” tubes, respectively. Then 0.1-ml of standards and quality controls in duplicate are added to the corresponding tubes. Next 0.1-ml of each plasma sample in duplicate is added to the “sample” tubes. This is followed by the addition of 0.1-ml of Sensitive Rat Insulin antibody (which also works well with mouse insulin) to all “sample” tubes. After mixing with a vortex apparatus, each sample is incubated at 4° C. for 24 hours.

On the second day, 0.1 ml of ¹²⁵I-insulin (˜1.4×10⁶ d.p.m.) is added to each tube (including 1-2 tubes) followed by mixing and incubating the covered tubes at 4° C. for 24 hours.

On the third day, 1.0-ml of cold (4° C.) precipitating reagent (supplied by the kit) is added to all tubes except tubes 1-2. After mixing and incubating for 20 min at 4° C., all tubes (except tubes 1-2) were centrifuged for 20 min at 3,000×g.

[Note: xg=(1.12×10⁻⁵) (r) (rpm)² where r=radial distance in cm (from axis of rotation to the bottom of the tube and rpm=rotational velocity of the centrifuge rotor].

Then the supernatant is immediately decanted (except tubes 1-2), the tubes are drained for about 60 sec, and excess liquid is blotted from the lip of tubes. This is followed by determination of remaining radioactivity (associated with immunprecipitate) in the tubes using a gamma counter. The amount of insulin in the mouse blood serum is determined by an automated data reduction procedure and expressed as ng/ml.

In the actual experiment, C57/Black female mice, weighing 22-23 g and fasted for 16 hours before being administered glucose intraperitoneally (3 g/kg) were used. Two hours prior to glucose load, the animals were intraperitoneally injected with aTF (0.5 mg per mouse), hTF (0.5 mg per mouse), or efTF (0.5 mg per mouse). Blood samples for insulin determination were taken from the eyes (canthus) with capillaries after 30 min of glucose administration. Each treatment group included five animals. The data are the mean±std. dev. of 5 determinations, i.e. one determination with each of the five animals. In a different set of mice (5 mice in each group), 30 minutes after glucose administration, blood samples were also taken to determine the effect of each test protein on the blood glucose level.

The results, shown in TABLE 7, indicate that each TF preparation significantly decreased blood glucose levels without increasing the level of circulating insulin. In fact, efTF, unlike the other preparations, decreased the amount of circulating insulin. While at this time it is not clear why efTF had a different effect than the other preparations, the important conclusion that can be drawn from the experiment is that TF likely modulates blood glucose level via increasing the sensitivity of target tissues to the actions of insulin.

TABLE 7
Various TF preparations do not increase blood insulin level.

| | Blood Glucose level (mM) | Blood insulin level (pg/ml) |
|---|---|---|
| Treatment | at 30 min | at 30 min |
| None | 1.4 ± 0.2 | 111 ± 26 |
| Glucose | 7.8 ± 0.5 | 279 ± 11 |
| aTF + Glucose | 3.4 ± 0.3 | 249 ± 53 |
| hTF + Glucose | 2.9 ± 0.4 | 279 ± 47 |
| efTF + Glucose | 3.2 ± 0.2 | 186 ± 29 |

### Example 9

### Determination of Possible Contamination of Transferrin Preparations by Insulin

Since commercial TF preparations, used in embodiments of the invention, are prepared from the human blood, it was important to test whether they contain insulin at any detectable level.

For the detection of insulin in the TF preparations, Linco's Ultra Sensitive Human Insulin Radioimmunoassay (RIA) Kit was used (LINCO Research, St. Charles, Mo. 63304, USA; catalog number: HI-11K). This kit provides about ten-times greater sensitivity compared to other commercially available Insulin RIA techniques and is designed for use when insulin concentrations are extremely low and/or when sample volumes are limited. It is a completely homologous assay because the antibody was raised against highly purified human insulin and both the standard and tracer are prepared using human insulin. This kit utilizes ¹²⁵I-labeled insulin and a sensitive insulin antiserum to determine the level of insulin in serum, plasma or tissue culture media by the double antibody/PEG (polyethylene glycol) technique.

Reagents: The following reagents are used for the technique:

Assay buffer—0.05M Phosphosaline pH 7.4 containing 0.025M EDTA (ethylenediamine tetraacetic acid), 0.0% sodium azide, and 1% RIA grade bovine serum albumine (BSA).

Human insulin antibody (sensitive)—Guinea pig anti-sensitive human insulin antibody in assay buffer.

HPLC purified lyophilized ¹²⁵I-Insulin (specific activity 367 μCi/μg).

Label hydrating buffer—assay buffer containing normal guinea pig serum as a carrier. (Used to hydrate ¹²⁵I-Insulin).

Insulin standards—purified recombinant insulin standards at the following concentrations: 0.2, 0.5, 1.0, 5.9, 10.0, and 20.0 μCi/ml.

Precipitating reagent—Goat anti-Guinea pig IgG serum, 3% PEG and 0.05% Triton X-100 in 0.05M Phosphosaline, 0.025M EDTA, and 0.08% sodium azide.

Assay Procedure:

Transfer 0.3, 0.2, and 0.1-ml of assay buffer to NSB tubes (3-4), reference (Bo) tubes, and to “sample” tubes, respectively. Then transfer either 0.1-ml of insulin standard and quality control solutions or 0.1-ml of human TF solutions (suspected source of human insulin) to the corresponding tubes. This is followed by the addition of 0.1-ml of Sensitive Human Insulin antibody to all tubes except “Total Count” count tubes (1-2) and NSB tubes (3-4). The samples are covered and incubated at room temperature for 24 hours. Then the ¹²⁵I-insulin tracer is hydrated with 27 ml of Label Hydrating Buffer and mixed; of this mixture 0.1-ml volume is transferred to all tubes followed by incubation of tubes at room temperature for 24 hours. Then, 1.0-ml of cold (4° C.) Precipitating Reagent is added to all tubes except “Total Count” tubes (1-2). The tubes are vortexed and incubated at 4° C. for 20 minutes. The tubes are centrifuged at 4° C. for 20 minutes at 3,000×g. The supernatant is then decanted, the tubes are drained for 60 seconds, and then the radioactivity associated with the pellets in the tubes was counted for 1 minute. The content of insulin in the transferrin samples was calculated by using an automated data reduction procedure as described in the kit's manual.

The presence of insulin was determined in each TF preparation tested in TABLE 7, each used at concentrations of 0.1 mg/ml, 0.25 mg/ml and 0.5 mg/ml. No detectable amount of insulin was found in any of the TF preparations. Since (according to our present knowledge) the only powerful glucose lowering substance in the blood is insulin, this experiment demonstrates that the effects of TF on the blood glucose level were not caused by contaminant insulin or any other potentially minor contaminant.

### Example 10

### Comparison of the Effects of Low Doses of aTF in the Absence or Presence of CrCl₃ on Blood Glucose Level in Glucose Tolerance Test

In this experiment, male C57BL/6 mice fasted for 16 hours prior to the glucose load (3 g/kg) were used. The test agents were administered via subcutaneous injection 2 hours prior to the glucose load. In the first group mice received only glucose. In the second group, mice were treated with only 0.1 μg chromium (III) chloride hexahydrate (CrCl₃; Sigma/Aldrich, catalog no. 23,072-3). In the third group, mice were treated with only 25 μg human apoTF (aTF) (available from Sigma-Aldrich, Inc., catalog number, T 2036 according to the 2004/2005 Sigma Catalog). In the fourth group, mice were treated with only 50 μg aTF. In the fifth group, mice were simultaneously treated with 0.1 μg CrCl₃ and 25 μg human aTF. In the sixth group, mice were simultaneously treated with 0.1 μg CrCl₃ and 50 μg human aTF. When used together, CrCl₃ and human aTF were present in the same solution (physiological saline).

The data, shown in TABLE 8, indicate that at the lower (25 μg), concentration of TF addition of CrCl₃ enhanced the effect of TF both after 30 and 60 minutes of glucose administration. The effect was somewhat less at the higher (50 μg) TF concentration, although the trend was the same. Overall, the data indicates that TF enhances glucose metabolism likely by enhancing uptake of chromium into insulin sensitive tissues.

TABLE 8
Relatively low doses of aTF combined with CrCl₃ efficiently
decrease blood glucose in glucose tolerance test.

| | Blood glucose level (mM) | | |
|---|---|---|---|
| Addition | 0 min | 30 min | 60 min |
| None | 1.6 ± 0.2 | 7.4 ± 0.4 | 4.8 ± 0.3 |
| CrCl₃ | 1.8 ± 0.4 | 7.1 ± 0.5 | 4.8 ± 0.4 |
| aTF, 25 μg | 1.5 ± 0.3 | 6.1 ± 0.3 | 4.4 ± 0.3 |
| aTF, 50 μg | 1.6 ± 0.4 | 4.0 ± 0.5 | 3.3 ± 0.2 |
| CrCl₃ + aTF, 25 μg | 1.8 ± 0.5 | 4.7 ± 0.4 | 3.5 ± 0.3 |
| CrCl₃ + aTF, 50 μg | 1.6 ± 0.3 | 3.1 ± 0.3 | 2.9 ± 0.2 |

1. A method of reducing the blood glucose level in a mammal with type 1 or type 2 diabetes comprising administering a therapeutically effective amount of transferrin, or an active derivative to the mammal.

2. The method of claim 1, wherein the transferrin is administered by an injection selected from intravenous, intraperitoneal, subcutaneous, intraarterial, intradermal, and intramuscular.

3. The method of claim 1, wherein the transferrin is prepared as an inhalation powder and administered via inhalation device.

4. The method of claim 1, wherein the transferrin is administered via a suitable minipump, inserted under the skin, allowing its controlled release.

5. The method of claim 1, wherein the therapeutically effective amount is about 0.05-1.0 g per m².

6. The method of claim 1, wherein the therapeutically effective amount is about 50-400 mg per 100 kg of the mammal's body mass.

7. The method of claim 1, further comprising co-administering an anti-diabetic medicament in combination with the transferrin or active derivative.

8. The method of claim 7, wherein the anti-diabetic medicament includes one of insulin, sulfonylureas, metiglinides, biguanides, inhibitors of α-glucosidase, thiazolidinediones, and derivatives of glucagon-like peptide or glucose-dependent insulinotropic polypeptide.

9. The method of claim 1, further including performing an insulin elevating procedure in combination with the transferrin or active derivative.

10. The method of claim 9, wherein the insulin elevating procedure includes transplantation of pancreas, islets, or other insulin-producing cells.

11. The method of claim 1, further comprising co-administering chromium or derivatives thereof

12. A treatment regimen for treating diabetes by periodically administering to a human a therapeutically effective amount of transferrin, or an active derivative.

13. A method of enhancing the survival of islet β-cells comprising contacting the islet β-cells with an effective amount of transferrin, or an active derivative.

14. The method of claim 13 wherein the transferrin contacts the islet cells in vivo.

15. The method of claim 13 wherein the transferrin contacts the islet cells in vitro.

16. The method of claim 13, further comprising contacting the islet β-cells with a promoter of islet survival including one of a growth factor, a thiazolidinedione, a derivative of glucagon-like peptide, betacellulin, or α₁-antitrypsin.

17. The method of claim 15, wherein the transferrin or active derivative is in the range of 0.1-50 μg per ml.

18. A method of reducing body weight loss induced by type 1 diabetic condition in a mammal by administering a therapeutically effective amount of transferrin, or an active derivative to the mammals.
